(19) United States
(12) Patent Application Publication
(10) Pub. No.: US 2012/0136232 A1
(43) Pub. Date: May 31, 2012

(54) NON-INTRUSIVE WEARABLE RESPIRATORY FAILURE ALARM APPARATUS AND METHOD THEREOF

(75) Inventors: Ji Wook JEONG, Daejeon (KR); Yong Won JANG, Daejeon (KR); Soo Yeul LEE, Daejeon (KR); Seung Hwan KIM, Daejeon (KR); Won Ick JANG, Daejeon (KR)

(73) Assignee: ELECTRONICS AND TELECOMMUNICATIONS RESEARCH INSTITUTE, Daejeon (KR)

(21) Appl. No.: 13/304,523

(22) Filed: Nov. 25, 2011

(30) Foreign Application Priority Data
Nov. 26, 2010 (KR) 10-2010-0118967

(51) Int. Cl. A61B 5/113; A61B 5/08; A61B 5/053

(52) U.S. Cl. 600/388; 600/386; 600/390

(57) ABSTRACT

A non-intrusive wearable respiratory failure alarm apparatus and method capable of detecting a user's respiratory failure state by estimating a change in a user's respiratory rate, while minimizing restrictions on a user's behavior are provided. The non-intrusive wearable respiratory failure alarm apparatus includes: a chest girth measurement unit including one or more resistive electro-threads attached to or worn on a user's chest, and generating a resistance value corresponding to the user's chest girth through the resistive electro-threads; a signal processing unit calculating a user's respiratory rate and whether or not the user is in respiratory failure by referring to the resistance value; and an output unit providing information regarding the user's respiratory rate and/or information regarding whether or not the user is in respiratory failure to the user or a monitoring device.

### CROSS-REFERENCE TO RELATED APPLICATIONS

This application claims the priority of Korean Patent Application No. 10-2010-0118967 filed on Nov. 26, 2010, in the Korean Intellectual Property Office, the disclosure of which is incorporated herein by reference.

### BACKGROUND OF THE INVENTION

1. Field of the Invention

The present invention relates to a non-intrusive wearable respiratory failure alarm apparatus and a method capable of detecting and warning of a user's respiratory failure state while minimizing restrictions on a user's behavior.

2. Description of the Related Art

When a respiratory bio-signal such as a respiratory rate is measured or a change in a respiratory function is measured, conventionally, inhalation and exhalation are directly externally collected from a human body, or inhalation and exhalation are allowed to pass through a filter to measure the velocity of a respiratory flow.

However, according such methods, external equipment must be used whenever a measurement is being performed, and the user (i.e., the measurement subject), must hold the device in his or her mouth and breathe during the measurement, causing user inconvenience.

### SUMMARY OF THE INVENTION

An aspect of the present invention provides a non-intrusive wearable respiratory failure alarm apparatus capable of estimating a change in a respiratory rate, while minimizing restrictions on a user's behavior, to detect and warn as to whether or not the user is in a respiratory failure state.

According to an aspect of the present invention, there is provided a non-intrusive wearable respiratory failure alarm apparatus including: a chest girth measurement unit including one or more resistive electro-threads attached to or worn on a user's chest, and generating a resistance value corresponding to the user's chest girth through the resistive electro-threads; a signal processing unit calculating a user's respiratory rate and whether or not the user is in respiratory failure by referring to the resistance value; and an output unit providing information regarding the user's respiratory rate and/or information regarding whether or not the user is in respiratory failure to the user or a monitoring device.

The output unit may include: a communication unit informing the monitoring device about one or more of information regarding the user's respiratory rate and information regarding whether or not the user is in respiratory failure; and a display unit informing the user about the user's respiratory rate and/or whether or not the user is in respiratory failure through one or more of characters, an image, light, sound, and vibrations.

The chest girth measurement unit may be implemented in the form of an external (or adhesive) contact, a band, or article of clothing which can be attached to or worn on the user's chest.

The signal processing unit may include: a resistance-voltage conversion unit converting the resistance value into a voltage value; an analog-to-digital (A/D) conversion unit analog-digital converting the voltage value to digitize the voltage value; a section capacity calculation unit calculating a section capacity of the user's chest by using the digitized voltage value; a respiratory rate calculation unit recognizing a variation of the section capacity of the user's chest by monitoring the section capacity of the user's chest, and recognizing a user's respiratory rate by using the variation of the section capacity of the user's chest; and a respiratory failure detection unit checking whether or not the user is in respiratory failure by monitoring the user's respiratory rate.

The respiratory rate calculation unit may have a function of recognizing a user's inspiratory volume and an expiratory volume by using the variation of the section capacity of the user's chest.

The resistance-voltage conversion unit may be matched to (or correspond to) the one or more resistive electro-threads, respectively, and may include one or more voltage distribution circuits varying a distributed amount of voltage according to a resistance value of the corresponding resistive electro-threads.

The communication unit may communicate with the monitoring device by using one of a wired communication, a wireless communication, and an infrared ray communication.

According to another aspect of the present invention, there is provided a respiratory failure alarm method including: recognizing a variation of a user's chest girth by using one or more resistive electro-threads attached to or worn on the user's chest; recognizing a respiratory rate from the variation of the user's chest girth; checking whether or not the user is in respiratory failure by monitoring the user's respiratory rate; and informing the user or a monitoring device about one of more of the user's respiratory rate and whether or not the user is in respiratory failure.

The method may further include: recognizing a respiratory rate from the variation in chest girth.

### BRIEF DESCRIPTION OF THE DRAWINGS

The above and other aspects, features and other advantages of the present invention will be more clearly understood from the following detailed description taken in conjunction with the accompanying drawings, in which:

FIG. 1A is a schematic block diagram of a respiratory failure alarm system according to an embodiment of the present invention;

FIG. 1B is a view showing a user wearing a non-intrusive wearable respiratory failure alarm apparatus according to an embodiment of the present invention;

FIG. 2 is a detailed block diagram of a signal processing unit according to an embodiment of the present invention;

FIG. 3 is a detailed block diagram of a monitoring device according to an embodiment of the present invention;

FIG. 4 is a flow chart illustrating a process of a non-intrusive wearable respiratory rate measuring method according to an embodiment of the present invention; and

FIG. 5 is a graph showing a change in a resistance value according to a user's respiratory behavior.

### DETAILED DESCRIPTION OF THE PREFERRED EMBODIMENT

Embodiments of the present invention will now be described in detail with reference to the accompanying drawings. The invention may, however, be embodied in many different forms and should not be construed as being limited to the embodiments set forth herein. Rather, these embodiments are provided so that this disclosure will be thorough and complete, and will fully convey the scope of the invention to those skilled in the art. In the drawings, the shapes and dimensions may be exaggerated for clarity, and the same reference numerals will be used throughout to designate the same or like components.

Throughout the specification and claims, unless explicitly described to the contrary, the word “comprise”, and variations such as “comprises” or “comprising”, will be understood to imply the inclusion of stated elements but not the exclusion of any other elements. While terms such as “ . . . , part”, “ . . . unit”, “module”, etc., described in the specification refer to a unit for processing at least one function or operation which may be implemented by hardware, software, or a combination thereof.

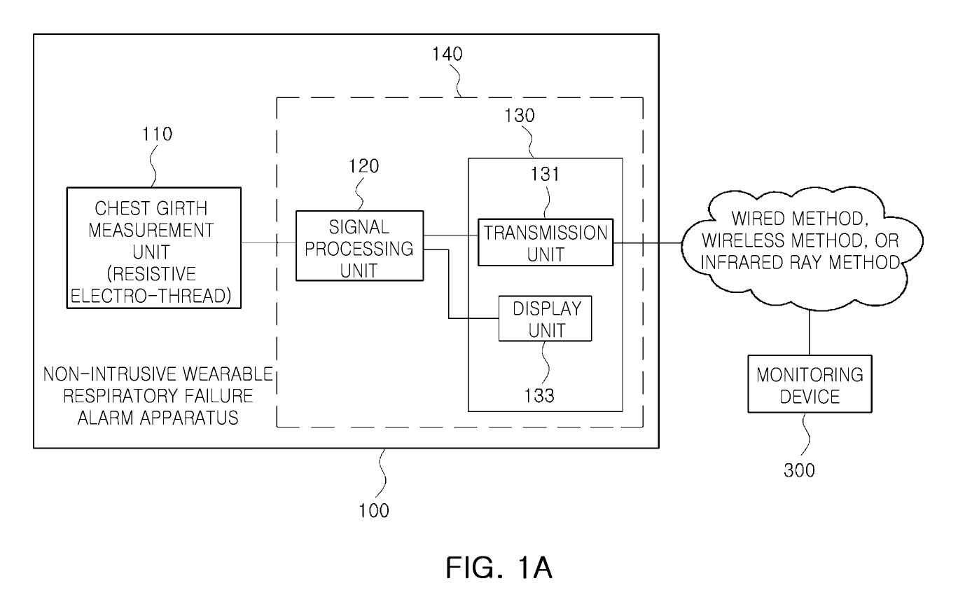

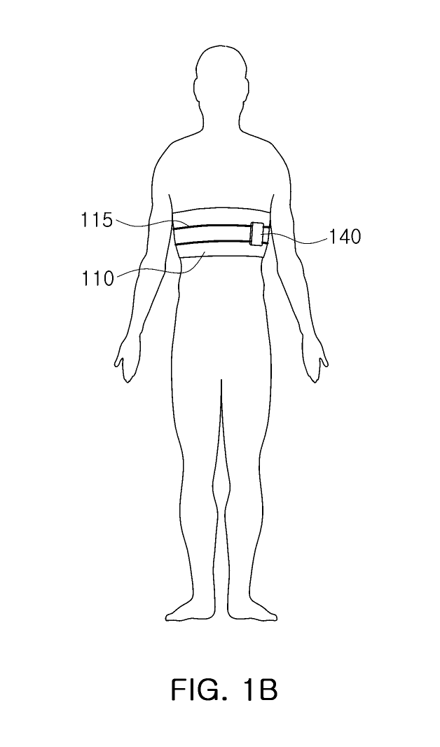

FIG. 1A is a schematic block diagram of a respiratory failure alarm system according to an embodiment of the present invention, and FIG. 1B is a view showing a user wearing a non-intrusive wearable respiratory failure alarm apparatus according to an embodiment of the present invention.

With reference to FIG. 1A, the respiratory failure alarm system may include a non-intrusive wearable respiratory failure alarm apparatus 100, a monitoring device 300, and the like, and the non-intrusive wearable respiratory failure alarm apparatus 100 may further include a chest girth measurement unit 110, a signal processing unit 120, an output unit 130, and the like. Here, the signal processing unit 120 and the output unit 130 may be implemented as a single hardware device 140.

The chest girth measurement unit 110 includes one or more resistive electro-threads 115 attached to or worn on a user's chest, and provides a resistance value corresponding to the user's chest girth through the resistive electro-threads 115.

The resistive electro-threads 115 include conductive yarn (or conductive fibers) and elastic yarn. When the user's chest girth is increased according to elasticity of the elastic yarn as the user breathes, the conductive yarn may be increased. Conductive yarn may include carbon fiber or a metal wire.

The resistive electro-thread 115 may be in a form in which conductive yarn and elastic yarn are twisted with each other. For example, conductive yarn may be twisted on and around elastic yarn, or conductive yarn may be doubly twisted on and around elastic yarn in mutually opposing directions.

With reference to FIG. 1B, the chest girth measurement unit 110 including a single or a plurality of resistive conductive yarns 115 is attached to or worn on a body part (e.g., the chest) which is most excessively changed in its sectional capacity (or bulk, volume) as the user breathes. The chest girth measurement unit 110 may be implemented in the form of an external (or adhesive) contact, a band, or the like, and independently put on, or may be woven into fabric so as to be implemented in the form of an article of clothing.

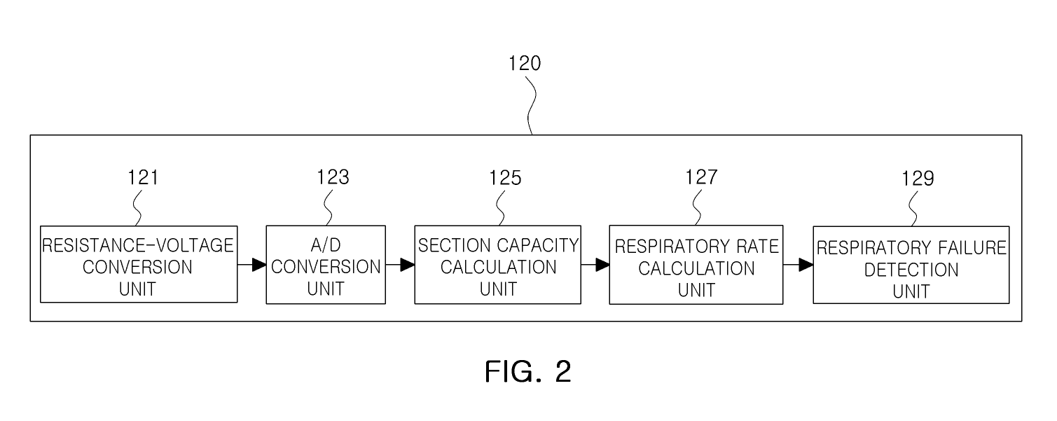

The signal processing unit 120 analyzes a resistance value provided by the chest girth measurement unit 110 to calculate the user's respiration rate, and monitors a change in the respiration rate to detect whether or not respiratory failure is taking place (i.e., whether or not the user is in respiratory failure). A detailed configuration of the signal processing unit 120 will be described later with reference to FIG. 2.

The output unit 130 includes a transmission unit 131 and a display unit 133.

The transmission unit 131 transmits information regarding respiration (e.g., respiration rate, whether or not respiratory failure is taking place (i.e., whether or not the user is in respiratory failure), or the like) obtained by the signal processing unit 120 and provided to the monitoring device 300. The transmission unit 131 may communicate with the monitoring device 300 by using one of a wired communication, a wireless communication, and an infrared ray communication.

The display unit 133, which includes various display devices such as a liquid crystal monitor, a light emitting element, a speaker, a vibration element, or the like, stimulates the user's sense of sight, sense of hearing, sense of touch, or the like, to inform the user about whether or not respiratory failure is taking place. The display unit 133 may also provide other information (i.e., the user's respiratory rate) to the user as well as information regarding whether or not a respiratory failure is taking place.

FIG. 2 is a detailed block diagram of a signal processing unit according to an embodiment of the present invention.

With reference to FIG. 2, the signal processing unit 120 may include a resistance-voltage conversion unit 121, an analog-to-digital (A/D) conversion unit 123, a section capacity calculation unit 125, a respiratory rate calculation unit 127, a respiratory failure detection unit 129, and the like.

The resistance-voltage conversion unit 121 converts the resistance value provided by the chest girth measurement unit 110 into a voltage value. To this end, the resistance-voltage conversion unit 121 may include one or more voltage distribution circuits which correspond to each of the resistive electro-threads 115 and varies a distributed amount of voltage according to a resistance value of the resistive electro-thread 115.

Each of the voltage distribution circuits may include a reference resistance and a variable resistance connected in series between a power voltage terminal and a ground terminal. The reference resistance is implemented as a general resistor element, while the variable resistance is implemented as the resistive electro-thread 115. When the user breathes, the girth of his chest is changed, and the length of the resistive electro-thread 115 is changed according to the change in the girth of the chest, so the resistive electro-thread 115 has a changed resistance value. Then, the size of voltage applied to the variable resistance (i.e., the resistive electro-thread 115) is changed. Thus, by measuring the size of the voltage, the resistance value can be converted into a voltage value.

The A/D conversion unit 123 converts an analog voltage value converted by the resistance-voltage conversion unit 121 into a digital value to digitize it. The A/D conversion unit 123 may include one or more A/D converters, each corresponding to each of the one or more voltage distribution circuits.

The section capacity calculation unit 125 calculates a section capacity of the user's chest by using the digital value digitized by the A/D conversion unit 123. When the user wears the respiratory failure alarm apparatus according to an embodiment of the present invention and inhales, the girth of his chest is increased, so the resistive electro-thread 115 elongates. When it is assumed that the user's body has a circular shape, the elongation of the resistive electro-thread 115 corresponds to an increase in the radius of the circle, so as the resistive electro-thread 115 elongates, the area of the circle is increased. Based on this principle, the section capacity calculation unit 125 can calculate a variation of the section capacity of the user's chest from the digital value from the A/D conversion unit 123.

The respiratory rate calculation unit 127 calculates a user's respiratory rate from the section capacity of the user's chest calculated by the section capacity calculation unit 125. Also, the respiratory rate calculation unit 127 may calculate an inspiratory volume and an expiratory volume by using the section capacity of the user's chest. For example, by recognizing an increment and a decrement of the section capacity of the user's chest, the inspiratory volume and expiratory volume of the user may be calculated, and by counting the number of peak value generation times of the section capacity value of the user's chest, the user's respiratory rate can be calculated. In this case, however, the peak value used for calculating the user's respiratory rate should have a value greater than a pre-set threshold value.

The respiratory failure detection unit 129 monitors a variation in the user's respiratory rate, and when the user's respiratory rate is sharply reduced and little respiration occurs (i.e., the user is scarcely breathing) even after the lapse of a certain period of time, the respiratory failure detection unit 129 confirms that a respiratory failure is taking place. Here, the respiratory failure detection unit 129 may previously receive a time used as a reference for determining the generation of the respiratory failure and the respiratory rate from the user.

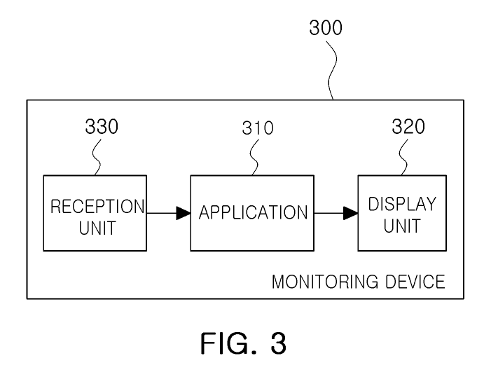

FIG. 3 is a detailed block diagram of a monitoring device according to an embodiment of the present invention.

With reference to FIG. 3, a monitoring device 300 may include an application 310, a display unit 320, and a reception unit 330. The monitoring device 300 may be implemented as a personal mobile terminal such as a mobile phone, a smartphone, or the like.

The application 310 performs various monitoring and reporting operations by using respiration-related information provided from the non-intrusive wearable respiratory failure alarm apparatus 100. For example, the application 310 may display the user's inspiratory volume and expiratory volume and the user's respiratory rate through a graphic user interface (GUI) and generate and immediately output information indicating that a respiratory failure is taking place to the user. Also, the application 310 may derive the user's physical condition from the user's inspiratory volume and expiratory volume, the user's respiratory rate, and whether or not respiratory failure is taking place, and provide the same to a hospital (e.g., a doctor), a health center, or the like.

The display unit 320 informs the holder of the monitoring device 300 about an output of the application 310 by using a display device provided in a personal mobile terminal. The output of the application 310 may be displayed in the form of characters, an image, light, sound, vibrations, or the like, which may stimulate the holder's sense of sight, sense of hearing, and sense of touch.

The reception unit 330 receives respiration-related information from the non-intrusive wearable respiratory failure alarm apparatus 100. The reception unit 330 of the monitoring device 300 may communicate with the non-intrusive wearable respiratory failure alarm apparatus 100 by using one of a wired communication, a wireless communication, and an infrared ray communication.

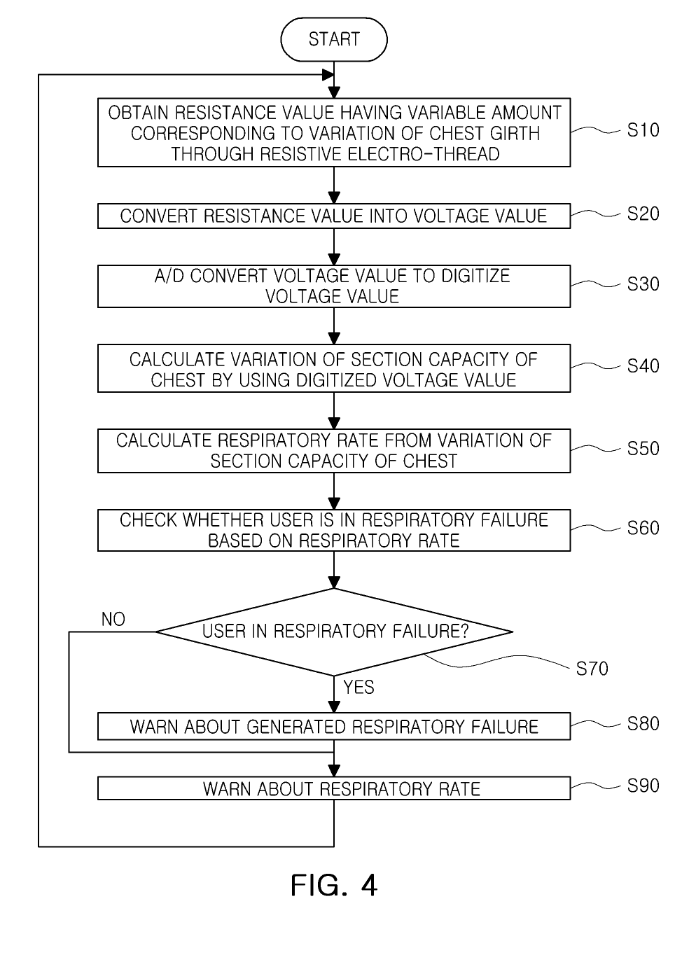

FIG. 4 is a flow chart illustrating a process of a non-intrusive wearable respiratory rate measuring method according to an embodiment of the present invention.

A specific embodiment of the non-intrusive wearable respiratory rate measuring method according to an embodiment of the present invention is the same as described above, so hereinafter, an operation process thereof will be briefly described.

With reference to FIG. 4, when the user breathes, a resistance value having a variable amount corresponding to a variation of the girth of the user's chest is obtained through the resistive electro-thread (S10).

The resistance value obtained in step S10 is converted into a voltage value (S20), and the converted voltage value is A/D-converted so as to be digitized (S30). A variation of the section capacity of the user's chest is calculated by using the digitized voltage value (S40).

While monitoring the variation of the section capacity of the user's chest, the number of generations of a peak value of the section capacity of the user's chest is counted to calculate the user's respiratory rate (S50), and whether or not a respiratory failure is taking place is checked based on the user's respiratory rate. Namely, when the user's respiratory rate is sharply reduced and such a state is maintained for more than a certain period of time, it is determined that a respiratory failure is taking place (S60).

When a respiratory failure is taking place according to the determination results in step S60 (S70), respiratory failure generation information is immediately displayed or provided to the monitoring device 300 (S80).

Also, when necessary, the obtained respiratory rate may be displayed or provided to the monitoring device 300 regardless of whether or not the respiratory failure is taking place (S90).

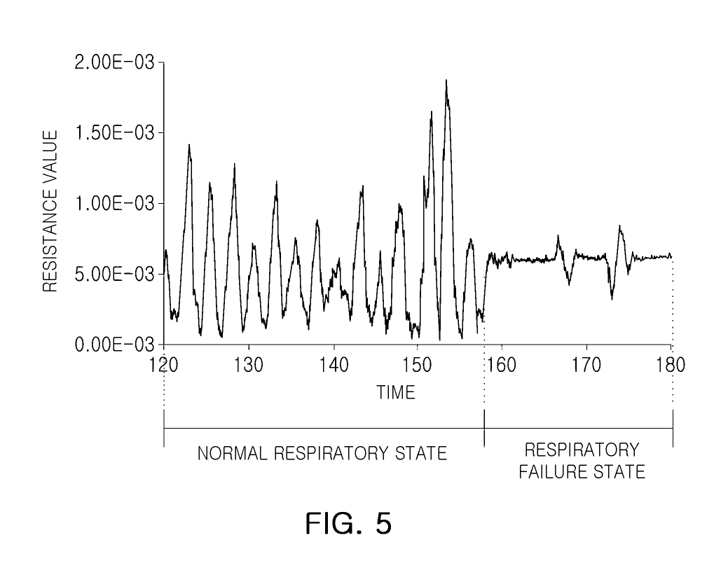

FIG. 5 is a graph showing a change in a resistance value according to a user's respiratory behavior.

As described above, a resistance value of the resistive electro-thread corresponds to a variation of the girth of the user's chest.

Thus, as shown in FIG. 5, it is noted that when the user breathes normally, the resistance value of the resistive electro-thread is repeatedly changed, while when the user is in a respiratory failure state, there is little change in the resistance value of the resistive electro-thread.

Thus, when the resistance value of the resistive electro-thread is not changed by more than a certain time (e.g., 30 seconds), it is determined that the user is in a respiratory failure state and the user or the monitoring device 300 is accordingly immediately informed, so that the user or the monitor of the monitoring device 300 can immediately take measures.

As set forth above, with the non-intrusive wearable respiratory failure alarm apparatus and method according to embodiments of the invention, a user's respiratory failure state can be detected and notified while minimizing restrictions on the user's behavior. Also, a change in the user's respiratory rate and whether or not a respiratory failure is taking place can be measured without external equipment or an external instrument.

In addition, continuous monitoring can be performed, and the user can be informed a change in the user's respiratory rate and whether or not a respiratory failure is taking place and respiratory-related information can be provided to a monitoring device through wired/wireless communication. Thus, when the user has a problem with respiratory behavior (e.g., an acute and chronic respiratory failure due to asthma or an allergy), information corresponding thereto can be provided to person other than the user, thus preventing an incident.

While the present invention has been shown and described in connection with the embodiments, it will be apparent to those skilled in the art that modifications and variations can be made without departing from the spirit and scope of the invention as defined by the appended claims.

What is claimed is:

1. A non-intrusive wearable respiratory failure alarm apparatus comprising:
a chest girth measurement unit including one or more resistive electro-threads attached to or worn on a user's chest, and generating a resistance value corresponding to the user's chest girth through the resistive electro-threads;
a signal processing unit calculating a user's respiratory rate and whether or not the user is in respiratory failure by referring to the resistance value; and
an output unit providing one or more of information regarding the user's respiratory rate and information regarding whether or not the user is in respiratory failure to the user or a monitoring device.

2. The apparatus of claim 1, wherein the output unit comprises:
a communication unit informing the monitoring device about one or more of information regarding the user's respiratory rate and information regarding whether or not the user is in respiratory failure; and
a display unit informing the user about one or more of the user's respiratory rate and/or whether or not the user is in respiratory failure by stimulating one or more of the user's sense of sight, sense of hearing, and sense of touch.

3. The apparatus of claim 1, wherein the chest girth measurement unit is implemented in the form of an external contact, a band, or an article of clothing which can be attached to or worn on the user's chest.

4. The apparatus of claim 1, wherein the signal processing unit comprises:
a resistance-voltage conversion unit converting the resistance value into a voltage value;
an analog-to-digital (A/D) conversion unit analog-digital converting the voltage value to digitize the voltage value;
a section capacity calculation unit calculating a section capacity of the user's chest by using the digitized voltage value;
a respiratory rate calculation unit recognizing a variation of the section capacity of the user's chest by monitoring the section capacity of the user's chest, and recognizing a user's respiratory rate by using the variation of the section capacity of the user's chest; and
a respiratory failure detection unit checking whether or not the user is in respiratory failure by monitoring the user's respiratory rate.

5. The apparatus of claim 4, wherein the user's respiratory rate calculation unit has a function of recognizing a user's inspiratory volume and an expiratory volume by using the variation of the section capacity of the user's chest.

6. The apparatus of claim 4, wherein the resistance-voltage conversion unit is matched to the one or more resistive electro-threads, respectively, and includes one or more voltage distribution circuits varying a distributed amount of voltage according to a resistance value of the corresponding resistive electro-threads.

7. The apparatus of claim 1, wherein the communication unit communicates with the monitoring device by using one of a wired communication, a wireless communication, and an infrared ray communication.

8. A respiratory failure alarm method comprising:
recognizing a variation of a user's chest girth by using one or more resistive electro-threads attached to or worn on the user's chest;
recognizing a respiratory rate from the variation of the user's chest girth;
checking whether or not the user is in respiratory failure by monitoring the user's respiratory rate; and
informing the user or a monitoring device about one or more of the user's respiratory rate and whether or not the user is in respiratory failure.

9. The method of claim 8, further comprising:
recognizing a respiratory rate from the variation in chest girth.
